(19) United States
(12) Patent Application Publication
(10) Pub. No.: US 2003/0219501 A1
(43) Pub. Date: Nov. 27, 2003

(54) All-natural antacid composition

(76) Inventor: Charles E. Day, Leitchfield, KY (US)

Correspondence Address: Dr. Charles E. Day, 1434 Sunbeam Road, Leitchfield, KY, 42754, US

(21) Appl. No.: 10/155,595

(22) Filed: May 24, 2002

(51) Int. Cl. A61K 35/78

(52) U.S. Cl. 424/765; 424/725

(57) ABSTRACT

Disclosed is an antacid composition comprising ellagitannin and glycine. The composition is useful to relieve gastric hyperacidity or gastroesophageal reflux disorder.

### FIELD OF THE INVENTION

The invention relates to an antacid composition. The invention also relates to the use of the composition in the relief of gastric hyperacidity or gastroesophageal reflux disorder.

### DESCRIPTION OF THE RELATED ART

Antacid compositions known in the art include many that are based on relatively few compounds, notably salts of calcium, magnesium, aluminum or bismuth. For example, U.S. Pat. No. 4,425,332 discloses a dosage form of finely divided aluminum hydroxide. More recently, U.S. Pat. No. 5,762,962 discloses compositions based on calcium or magnesium salts. The inventions disclosed in these two patents are representative of the art, which continues to rely in large measure on the use of a few metal salts. In addition, some citrates, tartrates and silicates are used as antacids, as are milk solids and the amino acid glycine. Regulations concerning over-the-counter antacid use are found in 21 C.F.R. § 331, which is incorporated by reference.

A major problem associated with the use of metallic antacids is their well-documented adverse interaction with widely-used drugs, including tetracycline and quinolone antibiotics, whose absorption is inhibited by cations present in widely-used antacids. Adverse interactions between antacids and various drugs are discussed in: Marchbanks CR (1993) Drug-drug interactions with fluoroquinolones. Pharmacotherapy 13, 23S; Gugler R & Allgayer H (1990) Effects of antacids on the clinical pharmacokinetics of drugs. Clin Pharmacokinet 18, 210; Bint A J & Burtt I (1980) Adverse antibiotic drug interactions. Drugs 20, 57. Moreover, many metallic antacids are not suitable for use by patients who present with kidney disease. Hatlebakk J G & Berstad A (1996) Pharmacokinetic optimisation in the treatment of gastro-oesophageal reflux disease. Clin Pharmacokinet 31, 386.

For treatment of underlying causes of the discomfort relieved by antacids, a number of small molecule pharmaceutical compositions have been developed. Among the best-known and most widely prescribed of such medications are the H-2 histamine receptor antagonists (e.g., U.S. Pat. No. 4,496,571) and the proton pump inhibitors (e.g., U.S. Pat. No. 5,677,302). Such agents do not in general impart immediate relief of the symptoms of gastric hyperacidity and gastroesophageal reflux. The use and mechanism of such agents is discussed in: Hirschowitz B I et al. (1995) Pharmacological aspects of acid secretion. Dig Dis Sci 40, 3S.

Although they offer effective therapy for many individuals, small molecule drugs used for the treatment of hypersecretory conditions exert certain undesired side-effects. Hatlebakk J G & Berstad A (1996) Pharmacokinetic optimisation in the treatment of gastro-oesophageal reflux disease. Clin Pharmacokinet 31, 386.

There has therefore been a need for a safe non-metallic antacid that imparts immediate and protracted relief of the hypersecretory conditions, gastric hyperacidity and gastroesophageal reflux.

The United States Food and Drug Administration lists parameters to which a composition comprising glycine must conform in order to be considered appropriate for labeling as an antacid. However, the art does not teach a composition comprising glycine that imparts immediate and protracted relief of hypersecretory conditions, gastric hyperacidity or gastroesophageal reflux.

Parietal cells which line the stomach produce hydrochloric acid. Romanes G J (1981) Cunningham's Textbook of Anatomy (12th ed.) at 450. Thus, parietal cells are the site of action of certain small molecule drugs used for the treatment of hypersecretory conditions.

The Physician's Desk Reference lists several antacids. Physicians' Desk Reference (54th ed. 2000) at 210, 786, 1465, 1466, 1992, 1993, and 3209. Each of the listed antacids contains one or more metal salts as active ingredients. Id. See also 21 C.F.R. § 331.11 (listing active ingredients for over-the-counter antacids and showing almost all to be metal salts).

Certain proton pump inhibitors, such as omeprazole, are known in the art to confer relief of gastric hyperacidity or gastroesophageal reflux disorder. The art teaches that the naturally occurring compound ellagic acid may also function as a proton pump inhibitor. Murakami et al. (1991) Inhibition of gastric H⁺,K⁺-ATPase and acid secretion by ellagic acid. Planta Med. 57, 305.

The art does not teach the efficacious use in a human subject of a composition comprising glycine and ellagic acid esters for the immediate and protracted relief of gastric hyperacidity or gastroesophageal reflux disorder. The literature lacks any report of a human subject experiencing immediate and protracted relief from an all-natural composition comprising glycine and ellagitannins.

### DESCRIPTION OF THE INVENTION

The invention provides inter alia an antacid composition comprising an ellagitannin and glycine.

The invention provides inter alia an antacid composition comprising an ellagitannin and glycine wherein the ellagitannin is derived from raspberry seed or strawberry seed.

The invention provides inter alia an antacid composition comprising an ellagitannin and glycine wherein the ellagitannin is derived from raspberry seed or strawberry seed and wherein the ellagitannin is derived from the seed by a process comprising comminuting the seed to form a plurality of seed particles.

The invention provides inter alia an antacid composition comprising an ellagitannin and glycine wherein the ellagitannin is derived from raspberry seed or strawberry seed and wherein the ellagitannin is derived from the seed by a process comprising contacting the seed with a fluid comprising water, an organic solvent, or supercritical carbon dioxide.

The invention provides inter alia an antacid composition comprising an ellagitannin and glycine wherein the ellagitannin is derived from raspberry seed or strawberry seed and wherein the ellagitannin is derived from the seed by a process comprising comminuting the seed to form a plurality of seed particles and wherein the ellagitannin is derived from the seed particles by a process comprising contacting at least one of the seed particles with a fluid comprising water, an organic solvent, or supercritical carbon dioxide.

The invention provides inter alia an antacid composition consisting essentially of ellagitannin, glycine, and a pharmaceutically acceptable carrier or diluent.

The invention provides inter alia an antacid composition consisting essentially of ellagitannin, glycine, and a pharmaceutically acceptable carrier or diluent wherein the ellagitannin is derived from raspberry seed or strawberry seed.

The invention provides inter alia an antacid composition consisting essentially of ellagitannin, glycine, and a pharmaceutically acceptable carrier or diluent wherein the ellagitannin is derived from raspberry seed or strawberry seed and wherein the ellagitannin is derived from the seed by a process comprising comminuting the seed to form a plurality of seed particles.

The invention provides inter alia an antacid composition consisting essentially of ellagitannin, glycine, and a pharmaceutically acceptable carrier or diluent wherein the ellagitannin is derived from raspberry seed or strawberry seed and wherein the ellagitannin is derived from the seed by a process comprising contacting the seed with a fluid comprising water, an organic solvent, or supercritical carbon dioxide.

The invention provides inter alia an antacid composition consisting essentially of ellagitannin, glycine, and a pharmaceutically acceptable carrier or diluent wherein the ellagitannin is derived from raspberry seed or strawberry seed and wherein the ellagitannin is derived from the seed by a process comprising comminuting the seed to form a plurality of seed particles and wherein the ellagitannin is derived from the seed particles by a process comprising contacting at least one of the seed particles with a fluid comprising water, an organic solvent, or supercritical carbon dioxide.

The invention provides inter alia an antacid composition consisting essentially of about 20%, by mass, extract of raspberry seed particles, and about 80%, by mass, glycine.

The invention provides inter alia an antacid composition consisting essentially of about 20%, by mass, extract of raspberry seed particles, and about 80%, by mass, glycine wherein the extract is derived from the raspberry seed particles by a process comprising contacting the raspberry seed particles with a fluid comprising water or acetone.

The invention provides inter alia a method of treating, lessening or preventing duodenal ulcer, gastric ulcer, gastroesophageal reflux disease, esophagitis, or pathological hypersecretory condition in a vertebrate comprising the step of administering to the vertebrate an effective amount of an antacid composition comprising an ellagitannin and glycine.

The invention provides inter alia a method of treating, lessening or preventing duodenal ulcer, gastric ulcer, gastroesophageal reflux disease, esophagitis, or pathological hypersecretory condition in a vertebrate comprising the step of administering to the vertebrate an effective amount of an antacid composition comprising an ellagitannin and glycine wherein the ellagitannin is derived from raspberry seed or strawberry seed.

The invention provides inter alia a method of treating, lessening or preventing duodenal ulcer, gastric ulcer, gastroesophageal reflux disease, esophagitis, or pathological hypersecretory condition in a vertebrate comprising the step of administering to the vertebrate an effective amount of an antacid composition comprising an ellagitannin and glycine wherein the ellagitannin is derived from raspberry seed or strawberry seed and wherein the ellagitannin is derived from the seed by a process comprising comminuting the seed to form a plurality of seed particles.

The invention provides inter alia a method of treating, lessening or preventing duodenal ulcer, gastric ulcer, gastroesophageal reflux disease, esophagitis, or pathological hypersecretory condition in a vertebrate comprising the step of administering to the vertebrate an effective amount of an antacid composition comprising an ellagitannin and glycine wherein the ellagitannin is derived from raspberry seed or strawberry seed and wherein the ellagitannin is derived from the seed by a process comprising contacting the seed with a fluid comprising water, an organic solvent, or supercritical carbon dioxide.

The invention provides inter alia a method of treating, lessening or preventing duodenal ulcer, gastric ulcer, gastroesophageal reflux disease, esophagitis, or pathological hypersecretory condition in a vertebrate comprising the step of administering to the vertebrate an effective amount of an antacid composition comprising an ellagitannin and glycine wherein the ellagitannin is derived from raspberry seed or strawberry seed and wherein the ellagitannin is derived from the seed by a process comprising comminuting the seed to form a plurality of seed particles and wherein the ellagitannin is derived from the seed particles by a process comprising contacting at least one of the seed particles with a fluid comprising water, an organic solvent, or supercritical carbon dioxide.

The invention provides inter alia a method of treating, lessening or preventing duodenal ulcer, gastric ulcer, gastroesophageal reflux disease, esophagitis, or pathological hypersecretory condition in a vertebrate comprising the step of administering to the vertebrate an effective amount of an antacid composition consisting essentially of ellagitannin, glycine, and a pharmaceutically acceptable carrier or diluent.

The invention provides inter alia a method of treating, lessening or preventing duodenal ulcer, gastric ulcer, gastroesophageal reflux disease, esophagitis, or pathological hypersecretory condition in a vertebrate comprising the step of administering to the vertebrate an effective amount of an antacid composition consisting essentially of ellagitannin, glycine, and a pharmaceutically acceptable carrier or diluent wherein the ellagitannin is derived from raspberry seed or strawberry seed.

The invention provides inter alia a method of treating, lessening or preventing duodenal ulcer, gastric ulcer, gastroesophageal reflux disease, esophagitis, or pathological hypersecretory condition in a vertebrate comprising the step of administering to the vertebrate an effective amount of an antacid composition consisting essentially of ellagitannin, glycine, and a pharmaceutically acceptable carrier or diluent wherein the ellagitannin is derived from raspberry seed or strawberry seed and wherein the ellagitannin is derived from the seed by a process comprising comminuting the seed to form a plurality of seed particles.

The invention provides inter alia a method of treating, lessening or preventing duodenal ulcer, gastric ulcer, gastroesophageal reflux disease, esophagitis, or pathological hypersecretory condition in a vertebrate comprising the step of administering to the vertebrate an effective amount of an antacid composition consisting essentially of ellagitannin, glycine, and a pharmaceutically acceptable carrier or diluent wherein the ellagitannin is derived from raspberry seed or strawberry seed and wherein the ellagitannin is derived from the seed by a process comprising contacting the seed with a fluid comprising water, an organic solvent, or supercritical carbon dioxide.

The invention provides inter alia a method of treating, lessening or preventing duodenal ulcer, gastric ulcer, gastroesophageal reflux disease, esophagitis, or pathological hypersecretory condition in a vertebrate comprising the step of administering to the vertebrate an effective amount of an antacid composition consisting essentially of ellagitannin, glycine, and a pharmaceutically acceptable carrier or diluent wherein the ellagitannin is derived from raspberry seed or strawberry seed and wherein the ellagitannin is derived from the seed by a process comprising comminuting the seed to form a plurality of seed particles and wherein the ellagitannin is derived from the seed particles by a process comprising contacting at least one of the seed particles with a fluid comprising water, an organic solvent, or supercritical carbon dioxide.

The invention provides inter alia a method of treating, lessening or preventing duodenal ulcer, gastric ulcer, gastroesophageal reflux disease, esophagitis, or pathological hypersecretory condition in a vertebrate comprising the step of administering to the vertebrate an effective amount of an antacid composition consisting essentially of about 20%, by mass, extract of raspberry seed particles, and about 80%, by mass, glycine.

The invention provides inter alia a method of treating, lessening or preventing duodenal ulcer, gastric ulcer, gastroesophageal reflux disease, esophagitis, or pathological hypersecretory condition in a vertebrate comprising the step of administering to the vertebrate an effective amount of an antacid composition consisting essentially of about 20%, by mass, extract of raspberry seed particles, and about 80%, by mass, glycine wherein the extract is derived from the raspberry seed particles by a process comprising contacting the raspberry seed particles with a fluid comprising water or acetone.

The invention relates to a composition comprising glycine and ellagitannin. One embodiment of the invention further comprises a pharmaceutically acceptable carrier or diluent. A preferred embodiment of the invention is a composition consisting of: 1% or less, by mass, raspberry ellagitannin; glycine; and a pharmaceutically acceptable carrier or diluent. A particularly preferred embodiment consists of an aqueous acetone extract of finely-macerated raspberry seed and glycine. The composition is administered perorally, preferentially one or more times daily, to a vertebrate, preferentially human, subject, to confer obviation of or relief from gastric hyperacidity or gastroesophageal reflux disorder.

As used in connection with the present invention, “comminuting” means acting upon a particle in such a way that the particle is divided into a plurality of smaller particles. By way of example and not of limitation, one may comminute a particle by grinding the particle, by pounding the particle, by subjecting the particle to shock waves, or by subjecting the particle to electromagnetic radiation. By way of example and not of limitation, a crude power resulting from the grinding of a seed is the result of the seed's being comminuted. As used in connection with the present invention, “derived” has its ordinary meaning in the art. For example, a crude powder resulting from the grinding of a seed is derived from the seed, or an extract of the crude powder is derived from the crude powder, and hence from the seed from which the crude powder is derived. As used in connection with the present invention, “organic solvent” means, by way of example and not of limitation, an aliphatic alcohol such as methanol, ethanol, or isopropanol, a ketone such as acetone, or, more generally, a substance known in the art to be an organic solvent. As used in connection with the present invention, the noun “extract” means a mixture resulting from contacting one or more particles with a fluid in such a way that at least a portion of one or more compositions initially present in the one or more particles enters, or is carried by or along with, the fluid, to form the mixture.

### DETAILED DESCRIPTION OF THE INVENTION

### EXAMPLE

Raspberry seeds were ground in a flour mill to form a crude powder. The crude powder was extracted with a solution that was, by volume, 80% acetone and 20% water, to form an extract. The solution was evaporated from the extract, leaving a berry acetone extract powder (BAEP) containing ellagitannins and adventitious matter. A mass of 0.4 grams of BAEP was mixed with a mass of 1.6 grams of glycine to form a BAEP-glycine mixture. The BAEP-glycine mixture was compressed in a hand tablet press to form a pressed tablet. One pressed tablet was taken each day by a human subject suffering from gastric hyperacidity who had customarily required at least one dose of omeprazole per day in order to experience relief of symptoms. Upon taking the pressed tablet, the subject experienced immediate and protracted relief from the subject's symptoms: the subject thereafter experienced relief from the symptoms of gastric hyperacidity by taking only one dose of omeprazole every second day during the period of time in which the subject was also taking at least BAEP-glycine tablet per day.

Further Examples. An additional example is prepared as described above in “Detailed Description of the Invention: Example” except that a pharmaceutically acceptable dose of omeprazole and a pharmaceutically acceptable carrier or diluent are mixed with BAEP and glycine and included in the pressed tablet.

Another example is prepared as described above in “Detailed Description of the Invention: Example” except that a pharmaceutically acceptable dose of the antacid polymer of polyethylenimine and aminopropylimidazole disclosed in U.S. Pat. No. 6,040,315 is mixed with BAEP and glycine and included in the pressed tablet.

Another additional example is prepared as described above in “Detailed Description of the Invention: Example” except that, instead of raspberry seeds, strawberry seeds are extracted.

Still another additional example is prepared as described above in “Detailed Description of the Invention: Example” except that a pharmaceutically acceptable dose of an antiflatulent is mixed with BAEP and glycine and included in the pressed tablet.

Yet another additional example is prepared as described above in “Detailed Description of the Invention: Example” except that a pharmaceutically acceptable dose of an H-2 receptor antagonist is mixed with BAEP and glycine and included in the pressed tablet.

Another additional example is prepared as described above in “Detailed Description of the Invention: Example” except that a pharmaceutically acceptable dose of a quinolone antibiotic is mixed with BAEP and glycine and included in the pressed tablet.

Another example is prepared as described above in “Detailed Description of the Invention: Example” except that a pharmaceutically acceptable dose of tetracycline is mixed with BAEP and glycine and included in the pressed tablet.

An additional example is prepared as described above in “Detailed Description of the Invention: Example” except that, instead of raspberry seeds, oak wood is extracted.

As another example, raspberry seeds were ground in a flour mill to form a crude powder. The crude powder was extracted with acetone to form an extract. The acetone was evaporated from the extract, leaving a berry acetone extract powder (BAEP) containing ellagitannins and adventitious matter. Such BAEP is useful as an antacid.

As another example, raspberry seeds were ground in a flour mill to form a crude powder. The crude powder was extracted with a solution that was, by volume, 90% acetone and 10% water, to form an extract. The solution was evaporated from the extract, leaving a berry acetone extract powder (BAEP) containing ellagitannins and adventitious matter. Such BAEP is useful as an antacid.

As another example, raspberry seeds were ground in a flour mill to form a crude powder. The crude powder was extracted with a solution that was, by volume, 70% acetone and 30% water, to form an extract. The solution was evaporated from the extract, leaving a berry acetone extract powder (BAEP) containing ellagitannins and adventitious matter. Such BAEP is useful as an antacid.

As another example, raspberry seeds were ground in a flour mill to form a crude powder. The crude powder was extracted with a solution that was, by volume, 60% acetone and 40% water, to form an extract. The solution was evaporated from the extract, leaving a berry acetone extract powder (BAEP) containing ellagitannins and adventitious matter. Such BAEP is useful as an antacid.

As another example, raspberry seeds were ground in a flour mill to form a crude powder. The crude powder was extracted with a solution that was, by volume, 50% acetone and 50% water, to form an extract. The solution was evaporated from the extract, leaving a berry acetone extract powder (BAEP) containing ellagitannins and adventitious matter. Such BAEP is useful as an antacid.

As another example, raspberry seeds were ground in a flour mill to form a crude powder. The crude powder was extracted with a solution that was, by volume, 40% acetone and 60% water, to form an extract. The solution was evaporated from the extract, leaving a berry acetone extract powder (BAEP) containing ellagitannins and adventitious matter. Such BAEP is useful as an antacid.

As another example, raspberry seeds were ground in a flour mill to form a crude powder. The crude powder was extracted with a solution that was, by volume, 30% acetone and 70% water, to form an extract. The solution was evaporated from the extract, leaving a berry acetone extract powder (BAEP) containing ellagitannins and adventitious matter. Such BAEP is useful as an antacid.

As another example, raspberry seeds were ground in a flour mill to form a crude powder. The crude powder was extracted with a solution that was, by volume, 20% acetone and 80% water, to form an extract. The solution was evaporated from the extract, leaving a berry acetone extract powder (BAEP) containing ellagitannins and adventitious matter. Such BAEP is useful as an antacid.

As another example, raspberry seeds were ground in a flour mill to form a crude powder. The crude powder was extracted with a solution that was, by volume, 10% acetone and 90% water, to form an extract. The solution was evaporated from the extract, leaving a berry acetone extract powder (BAEP) containing ellagitannins and adventitious matter. Such BAEP is useful as an antacid.

As another example, raspberry seeds were ground in a flour mill to form a crude powder. The crude powder was extracted with water to form an extract. Water was evaporated from the extract, leaving a berry acetone extract powder (BAEP) containing ellagitannins and adventitious matter. Such BAEP is useful as an antacid.

As another example, raspberry or strawberry seeds are ground in a flour mill, or commutated, or pounded, to form a crude powder. The crude powder is extracted with a fluid comprising ethanol, methanol, isopropanol, or supercritical carbon dioxide, to form an extract. The fluid is evaporated from the extract, leaving a berry acetone extract powder (BAEP) containing ellagitannins and adventitious matter. Such BAEP is useful as an antacid.

It is to be understood that the invention is not to be limited to the exact details of operation, or to the exact compositions, methods, procedures, or embodiments shown and described, as obvious modifications and equivalents will be apparent to one skilled in the art, and the invention is therefore to be limited only by the full scope which can fairly, legally and equitably be accorded to the appended claims.

I claim:

1. An antacid composition comprising an ellagitannin and glycine.

2. The composition of claim 1 wherein the ellagitannin is derived from raspberry seed or strawberry seed.

3. The composition of claim 2 wherein the ellagitannin is derived from the seed by a process comprising comminuting the seed to form a plurality of seed particles.

4. The composition of claim 2 wherein the ellagitannin is derived from the seed by a process comprising contacting the seed with a fluid comprising water, an organic solvent, or supercritical carbon dioxide.

5. The composition of claim 3 wherein the ellagitannin is derived from the seed particles by a process comprising contacting at least one of the seed particles with a fluid comprising water, an organic solvent, or supercritical carbon dioxide.

6. An antacid composition consisting essentially of ellagitannin, glycine, and a pharmaceutically acceptable carrier or diluent.

7. The antacid composition of claim 6 wherein the ellagitannin is derived from raspberry seed or strawberry seed.

8. The composition of claim 7 wherein the ellagitannin is derived from the seed by a process comprising comminuting the seed to form a plurality of seed particles.

9. The composition of claim 7 wherein the ellagitannin is derived from the seed by a process comprising contacting the seed with a fluid comprising water, an organic solvent, or supercritical carbon dioxide.

10. The composition of claim 8 wherein the ellagitannin is derived from the seed particles by a process comprising contacting at least one of the seed particles with a fluid comprising water, an organic solvent, or supercritical carbon dioxide.

11. An antacid composition consisting essentially of about 20%, by mass, extract of raspberry seed particles, and about 80%, by mass, glycine.

12. The composition of claim 11 wherein the extract is derived from the raspberry seed particles by a process comprising contacting the raspberry seed particles with a fluid comprising water or acetone.

13. A method of treating, lessening or preventing duodenal ulcer, gastric ulcer, gastroesophageal reflux disease, esophagitis, or pathological hypersecretory condition in a vertebrate comprising the step of administering to the vertebrate an effective amount of the composition of claim 1.

14. A method of treating, lessening or preventing duodenal ulcer, gastric ulcer, gastroesophageal reflux disease, esophagitis, or pathological hypersecretory condition in a vertebrate comprising the step of administering to the vertebrate an effective amount of the composition of claim 2.

15. A method of treating, lessening or preventing duodenal ulcer, gastric ulcer, gastroesophageal reflux disease, esophagitis, or pathological hypersecretory condition in a vertebrate comprising the step of administering to the vertebrate an effective amount of the composition of claim 3.

16. A method of treating, lessening or preventing duodenal ulcer, gastric ulcer, gastroesophageal reflux disease, esophagitis, or pathological hypersecretory condition in a vertebrate comprising the step of administering to the vertebrate an effective amount of the composition of claim 4.

17. A method of treating, lessening or preventing duodenal ulcer, gastric ulcer, gastroesophageal reflux disease, esophagitis, or pathological hypersecretory condition in a vertebrate comprising the step of administering to the vertebrate an effective amount of the composition of claim 5.

18. A method of treating, lessening or preventing duodenal ulcer, gastric ulcer, gastroesophageal reflux disease, esophagitis, or pathological hypersecretory condition in a vertebrate comprising the step of administering to the vertebrate an effective amount of the composition of claim 6.

19. A method of treating, lessening or preventing duodenal ulcer, gastric ulcer, gastroesophageal reflux disease, esophagitis, or pathological hypersecretory condition in a vertebrate comprising the step of administering to the vertebrate an effective amount of the composition of claim 7.

20. A method of treating, lessening or preventing duodenal ulcer, gastric ulcer, gastroesophageal reflux disease, esophagitis, or pathological hypersecretory condition in a vertebrate comprising the step of administering to the vertebrate an effective amount of the composition of claim 8.

21. A method of treating, lessening or preventing duodenal ulcer, gastric ulcer, gastroesophageal reflux disease, esophagitis, or pathological hypersecretory condition in a vertebrate comprising the step of administering to the vertebrate an effective amount of the composition of claim 9.

22. A method of treating, lessening or preventing duodenal ulcer, gastric ulcer, gastroesophageal reflux disease, esophagitis, or pathological hypersecretory condition in a vertebrate comprising the step of administering to the vertebrate an effective amount of the composition of claim 10.

23. A method of treating, lessening or preventing duodenal ulcer, gastric ulcer, gastroesophageal reflux disease, esophagitis, or pathological hypersecretory condition in a vertebrate comprising the step of administering to the vertebrate an effective amount of the composition of claim 11.

24. A method of treating, lessening or preventing duodenal ulcer, gastric ulcer, gastroesophageal reflux disease, esophagitis, or pathological hypersecretory condition in a vertebrate comprising the step of administering to the vertebrate an effective amount of the composition of claim 12.
